(19) United States
(12) Patent Application Publication
(10) Pub. No.: US 2004/0186457 A1
(43) Pub. Date: Sep. 23, 2004

(54) Needleless access vial

(76) Inventors: Timothy Lee Truitt, Orange, CA (US); Alex T. Mazza, Grand Terrace, CA (US); Raymond P. Feith, Chino Hills, CA (US)

Correspondence Address: JONES DAY, 222 EAST 41ST ST, NEW YORK, NY, 10017, US

(21) Appl. No.: 10/390,140

(22) Filed: Mar. 17, 2003

(51) Int. Cl. A61B 19/00

(52) U.S. Cl. 604/403

(57) ABSTRACT

A fluid flow control device adapted to control the flow of fluid from a fluid container to a fluid extraction device without the need for a needle. The fluid flow control device includes a connector element and a container insert member which define a fluid channel. A compressible valve element is disposed in the fluid channel of the connector element and the container insert member. The valve element includes a plug and shaft and resiliently prevents the flow of fluid by forming a fluid seal in the fluid channel. The valve element may be operationally engaged by the fluid extraction device thereby breaking the fluid seal and allowing the fluid to flow from the container to the fluid extraction device.

### FIELD OF THE INVENTION

The present invention relates to flow control and more particularly, to the control of fluid flow from containers of medical solutions through valves, access parts, and other connectors.

### BACKGROUND OF THE INVENTION

When it is contemplated that a patient will require the administration of fluid medicament, a medicament delivery apparatus, such as a syringe having a needle, and a medicament container are commonly required. Currently, most medicament containers are typically a fluid vial or container that is sealed with a rubber elastomer or septum. To access the contents of the container or vial, the rubber elastomer or septum must be pierced with a needle or similarly sharp instrument. Once the elastomer or septum is pierced, the contents of the container or vial can be accessed for administration to the patient. However, there are several drawback to using this type of container or vial.

First, using a container or vial that requires the use of a needle or sharp instrument for access to the contents of the container/vial creates a hazardous working environment for a healthcare worker. Not only is the healthcare worker exposed to possible bodily injury via an accidental needle stick, but once the needle has been used to administer the fluid or medicament to the patient, the needle is also considered a high risk article. Thus, the healthcare worker may also be exposed to potential diseases and other life threatening diseases from the high risk needle.

Second, once the septum or elastomer is pierced, the medicament in the container or vial may leak since these containers or vials are not completely resealable. Also, there is a possibility that bacteria may develop in and around the pierced septum, tainting the remaining contents of the container or vial. This reduces the desirability of using the container or vial as a multiple dose container.

Third, the container or vial can be particularly difficult to manipulate to access the entire contents of the container, especially in situations where there is only a small amount of medicament or fluid left in the container or vial.

Fourth, the container or vial is not compatible with some fluid delivery systems. Currently, there are needleless fluid delivery systems that allow access to the patient's vascular system via the connector of an intravenous tubing set. For example, U.S. Pat. No. 5,782,816 to Werschmidt et. al., the entire contents of which is incorporated herein by reference, discloses a bi-directional valve that allows access to a connector without the need for a needle. Transferring the contents from the container or vial to the needleless system would be difficult since the needleless system uses a connector that is not designed to be accessed by a needle, while the container or vial requires a needle to access its contents. Accordingly, to transfer the contents from the container or vial to a needleless system, a user must first access the contents with a needle, transfer the contents to a second instrument that does not have a needle, and, via the second instrument, transfer the contents to the needless system.

Therefore, there exists a need for a container or vial that does not require a needle to access its contents. There also exists a need for a container or vial that is resealable. There further exists a need for a container or vial that is easy to manipulate to allow quick and easy access to its contents. There also exists a need for a container that is compatible for use with a needleless system. There further exists a need for a container or vial that easily allows access to its entire contents.

### SUMMARY OF THE INVENTION

The present invention relates to a fluid control device for use with a fluid container. The device allows for needleless access of the contents stored in the fluid container and is compatible for use with other needleless systems. The device also is resealable and is easily manipulable to allow quick and easy access to substantially the entire contents of the container. Furthermore, the device provides a swabable surface to allow for easy cleaning and decontamination.

In one embodiment, the fluid flow control device includes a container insert member, a connector element defining a valve seat coupled to the container insert member, and a resilient valve element disposed in the container insert element and the connector element, and being movable from a first position to a second position. The valve element, in its first position, forms a seal with the valve seat. The valve element, in its second position, is canted, bent, buckled, bowed, or moved from the valve seat to create a fluid channel.

The container insert member can have a hollow interior defining an opening extending from a proximal surface of the container insert member to the interior of the container insert member where at least one window extends from an outer surface of the container insert member to the interior of the member and extends along the container insert member from the distal end to the proximal end of the container insert member. The container insert member may also have a flange located near the proximal end of the container insert member for abutting a lip surrounding a fluid container opening.

The connector element has an axial throughbore, a portion of which forms a valve seat, extending from the proximal end to the distal end of the connector element and further includes a collar located at the distal end of the connector element for coupling to the proximal end of the container insert member. The connector element may be configured for coupling with a fluid extraction device near its proximal end. For example, the fluid extraction device may have a male Luer fitting for coupling to the proximal end of the connector element. The coupling of the male Luer fitting to the proximal end of the connector element moves the valve element from the first position to the second position, thereby forming a fluid channel between the fluid container and the male Luer fitting of the fluid extraction device.

The valve element includes a plug disposed near the proximal end of the connector element, and a shaft formed at the distal end of the plug and extending to the distal end of the container insert member. The distal end of the valve element abuts and rests on the distal end of the interior surface of the container insert member. The plug and the shaft of the valve element may have at least one buckling section with characteristics for being axially compressible to move the valve element from the first position to the second position.

The fluid control device may also include a container wrapper member configured and dimensioned to assist in coupling the flange of the container insert member to the container.

### BRIEF DESCRIPTION OF THE DRAWINGS

To facilitate an understanding of and for the purpose of illustrating the present invention, exemplary and preferred features and embodiments are disclosed in the accompanying drawings, it being understood, however, that the invention is not limited to the precise arrangements and instrumentalities shown, and wherein similar reference characters denote similar elements throughout the several views, and wherein:

FIG. 1 is an exploded view of a fluid flow control device with a fluid container according to the principles of the present invention;

FIG. 2 is a perspective view of the device of FIG. 1 assembled;

FIG. 3 is a perspective view of a connector element of the fluid flow control device of FIG. 1;

FIG. 4 is a bottom view of the connector element of FIG. 3;

FIG. 5 is a perspective view of a container insert element of the fluid flow control device of FIG. 1;

FIG. 6 is a side cross-sectional view of an exemplary fluid flow control device in accordance with the principles of the present invention with the fluid container of FIG. 1 in a closed state;

FIG. 7 is a side cross-sectional view of the fluid flow control device of FIG. 7 activated by a male Luer fitting to an open state; and

FIG. 8 is a perspective view of a container wrapper member of the fluid flow control device of FIG. 1.

### DETAILED DESCRIPTION OF THE INVENTION

Referring to the accompanying drawings, preferred embodiments and features of the fluid flow control device for a fluid container will be described in detail. It is to be noted however that these descriptions of specific embodiments and features are merely illustrative. It is contemplated that one or more features or elements of the various embodiments may be combined or used singularly, and that modifications of the various embodiments, as well as other embodiments, are contemplated and will be apparent to those persons skilled in the art.

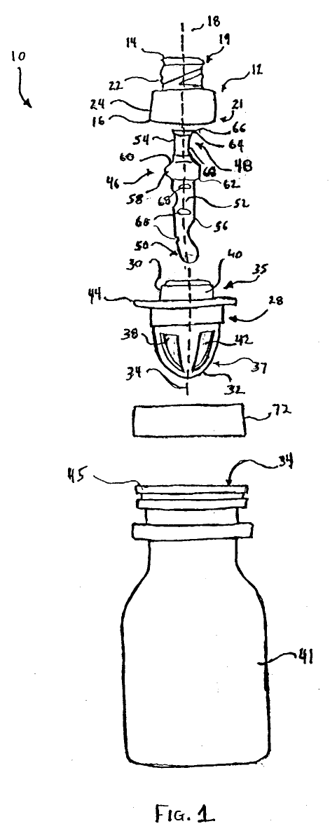

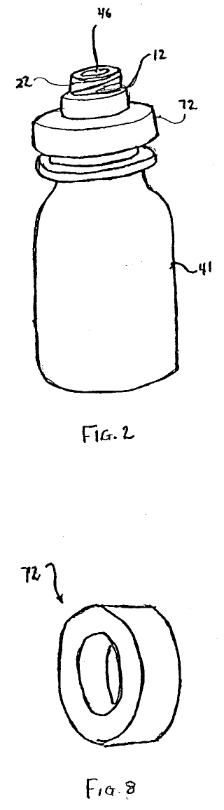

Referring initially to FIG. 1, an exploded view of an exemplary embodiment of fluid control device 10 is shown. Control device 10 preferably has a connector element 12 having proximal end 14, distal end 16, and longitudinal axis 18. In the embodiments of FIGS. 1, 2, 3, and 4, connector element 12 has a generally circular-cylindrical shape with an axial throughbore 20, which extends along axis 18 from proximal end 14 to distal end 16. Proximal portion 19 of connector element 12 is configured and dimensioned to interface with a fitting, preferably needleless, such as a male Luer fitting, from a fluid extraction device, such as a syringe or intravenous tubing. Although shown as circular-cylindrical, connector element 12 may be any shape that allows connection to a fluid extraction device and to the container insert member. For example, connector element 12 may be hexagonally-cylindrical or octagonally-cylindrical. In an exemplary embodiment, proximal portion 19 of connector element 12 may also include threading 22 on an exterior surface for threadably engaging the fitting of the fluid extraction device. Threading 22 allows the fitting to engage connector element 12 securely, thereby preventing accidental disconnection or fluid leakage. Distal portion 21 of connector element 12 may have an enlarged diameter section, or collar 24, for facilitating coupling of connector element 12 to a container insert member, discussed below. In the embodiment illustrated, the portion of axial throughbore 20 located in distal portion 21 has a larger diameter than the portion of axial throughbore 20 located in proximal portion 19.

Turning to FIG. 4, a bottom view of connector element 12 is shown. In an exemplary embodiment, a portion of axial bore 20 forms valve seat 26. Valve seat 26 is sized to receive a portion of a valve element (element 46 discussed below) in sealing engagement to form a primary fluid seal. Preferably, valve seat 26 includes at least one sealing surface 27, which is a transitionary surface in which axial bore 20 increases in diameter from the smaller diameter section located in proximal portion 19 to the larger diameter section located in collar 24 in distal portion 21. Although valve seat 26 is shown to be located near the proximal end of collar 24 of connector element 12, valve seat 26 may be located any place along axial bore 20.

Referring back to FIG. 1, control device 10 also preferably includes a container insert member 28. Container insert member 28 has a proximal end 30, a distal end 32, and a longitudinal axis 34. Proximal portion 35 is configured and sized to interface and to couple with connector element 12 and distal portion 37 is configured and sized to fit within fluid container 41 via opening 39. Fluid container 41 may be any container capable of storing fluid, such as medicament, and having at least one opening or access point through which the contents inside the container may be accessed.

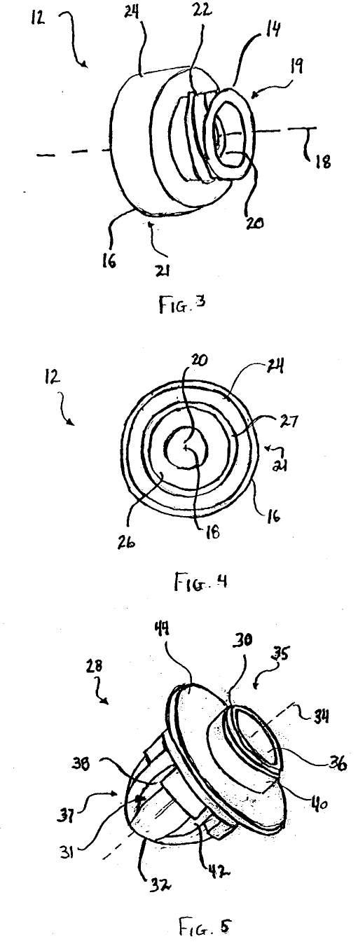

As can be seen in FIGS. 1 and 5, at proximal end 30, container insert member 28 has opening 36 which leads to hollow interior 38. In an exemplary embodiment, surrounding opening 36 is collar 40. Collar 40 may be configured and dimensioned to be coupled within collar 24 on connector element 12 and thereby to be coupled to fluid container 41. Preferably, the outer diameter of collar 40 is substantially equal to the inner diameter of axial throughbore 20 at distal portion 21 of connector element 12, allowing collar 40 to be received in and coupled to collar 24. Collar 24 and collar 40, and thus connector element 12 and container insert member 28, may be coupled together in a number of ways including, without limitation, bonding agents, ultrasonic welding, interference fitting, and tongue and groove fittings. Preferably, this coupling prevents leakage of any fluid. When coupled together, axial bore 20 of connector element 12 is in fluid communication with opening 36 and interior 38 of container insert member 28, creating a channel which extends from proximal end 14 of connector element 12 to distal end 32 of container insert member 28.

As mentioned above, distal portion 37 of container insert member 28 is received in fluid container 41. In an exemplary embodiment, distal portion 37 is dimensioned so that its outer diameter is substantially equal to or less than the inner diameter of opening 39 of container 41 allowing distal portion 37 to be received in container 41. Opening 39 and distal portion 37, and thus container 41 and container insert member 28, may be coupled together in a number of ways, including, without limitation, bonding agents, ultrasonic welding, tongue and groove fittings, and interference fitting. Preferably, this coupling prevents leakage of any fluid. In an alternate embodiment, connector element 12 and container insert member 28 may be portions of a one-piece housing or connector.

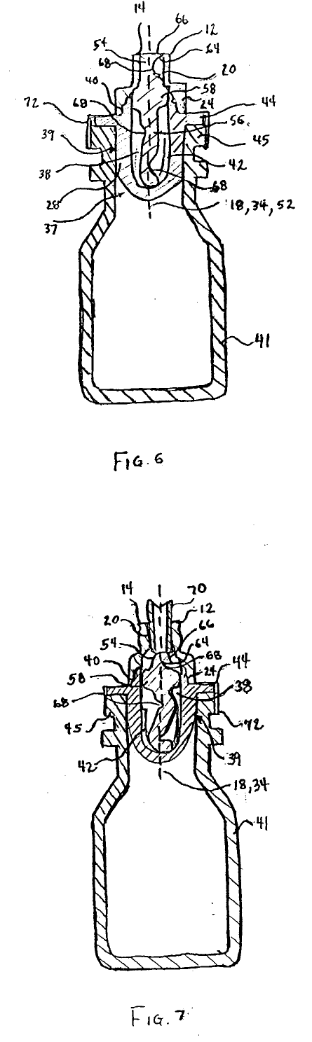

Distal portion 37 of insert member 28 has at least one window 42 therethrough, thereby fluidly connecting the interior of container 41 with interior 38. In an exemplary embodiment, there are a plurality of windows 42 (e.g., four), spaced an equal distance apart from one another and extending around distal portion 37. Windows 42 extend from distal end 32 to proximal portion 35 and are sized and spaced to maximize fluid flow from container 41 into interior 38 and to minimize remnant fluid in container 41. In an exemplary embodiment, windows 42 extend substantially the entire length of distal portion 37 of container insert member 28. As can be seen more clearly in FIGS. 6 and 7, windows 42 are arranged, configured, and dimensioned so that fluid in container 41 cannot pool around container insert member 28 if fluid container 41 is inverted since there is no wall or similar structure to impede the fluid flow. Rather, substantially all of the fluid in container 41 can and will flow into interior 38 through windows 42, thereby minimizing remnant fluid in container 41.

Distal end 32 of container insert member 28 also may have a rounded exterior surface. A corresponding portion of interior 38, located near distal end 32, may also be rounded. A rounded distal end 32 further minimizes remnant fluid in container 41. If container 41 is inverted, the fluid inside container 35 cannot pool on any outer surface of distal end 32 of container insert member 28. Instead, the fluid will flow down the rounded outer surface and into interior 38 through windows 42.

In a preferred embodiment, container insert member 28 also has a radially extending flange 44 located near proximal end 30. Flange 44 serves a two-fold purpose. First, flange 44 abuts lip 45 surrounding opening 39 of fluid container 41 to limit the insertion depth of container insert member 28. Preferably, flange 44 is located near proximal portion 35 so that container insert member 28 can be inserted into the fluid container to a depth that exposes a substantial portion of each of windows 42 to the interior of container 41 while also leaving a collar 40 of sufficient length for coupling connector element 12 thereto. Second, flange 44 serves as a coupling surface to couple container insert member 28 to fluid container 41. Flange 44 can be coupled to fluid container 41 in a number of ways, including, without limitation, via bonding agents, ultrasonic welding, tongue and groove fittings, and interference fitting. In an exemplary embodiment, an “o”-ring may also be included between flange 44 and container 41 to create a fluid seal and to further assist with coupling container insert member 28 to fluid container 41, particularly if a glass fluid container is used.

Referring back to FIG. 1, control device 10 also includes a valve element 46. Valve element 46 has a proximal end 48, a distal end 50, and a longitudinal axis 52. Valve element 46 is disposed within axial bore 20 of connector element 12 and interior 38 of container insert member 28. In an exemplary embodiment, valve element 46 is supported by interior 38 of container insert member 28. Particularly, distal end 50 of valve element 46 abuts the distal portion 31 of interior 38 of container insert member 28 such that valve element 46 may thereby be supported by the distal portion of interior 38. As shown in FIGS. 1 and 6, valve element 46 is, when in a neutral, sealing position, (defined below) substantially coaxial with connector element 12 and container insert member 28. Accordingly, axes 18, 34, and 52 are substantially collinear when valve element 46 is in the neutral, sealing position.

In an exemplary embodiment, valve element 46 may be formed partially or totally of elastomeric materials to facilitate deflection and sealing characteristics. In such embodiment, valve element 46 preferably includes a plug portion 54 and a shaft portion 56 extending in a distal direction from plug 54. Plug portion 54 includes a flange portion 58 having a proximal surface 60 and a distal surface 62. Proximally of flange portion 58, plug portion 54 has a generally cylindrical configuration and extends to nose 64 having a proximal end surface 66. Between proximal end surface 66 of nose 64 and proximal surface 60 of flange portion 58, plug portion 54 is provided with a buckling section. Such section may be at a predetermined location laterally of axis 52 to facilitate non-axial buckling of such section. In an exemplary embodiment, this buckling section may be a notch 68. As can be seen in FIGS. 1, 6, and 7, the illustrated exemplary valve element 46 has a plurality of buckling sections or notches 68 located on plug portion 54 and shaft portion 56 to facilitate bending, buckling, bowing or canting of valve element 46 as detailed below. Also shown in FIGS. 1, 6, and 7, the distal end 57 of shaft portion 56 may be rounded. This rounded end, which abuts and rests against the rounded distal portion 31 of interior 38 of container insert member 28, also facilitates bending, buckling, bowing or canting of valve element 46.

Valve element 46 enables fluid control device 10 to provide a neutral, sealing position in one operative state, and to provide an actuated, open position in a second operative state. The closed condition or state is illustrated in FIG. 6 where proximal end surface 66 is substantially coplanar with distal end 14 of connector element 12; the open condition or state is illustrated in FIG. 7, where proximal end surface 66 is no longer coplanar with distal end 14 of connector element 12. For the sake of convenience and with no intent to limit, the closed condition or state will be referred to as the closed state and the open condition or state will be referred to as the open state.

In the closed state, valve element 46 is generally in a neutral, sealing position and is substantially its maximum length. In this closed state, plug portion 54, associated with the valve element 46, is in sealing engagement with axial bore 20 of connector element 12. More specifically, proximal surface 60 of flange 58 is pressed into sealing engagement with valve seat 27 to form a primary seal between connector element 12 and plug portion 54 of valve element 46. In the closed state of valve element 46, a secondary seal is also formed between the circumferential surface of nose 64 and axial bore 20 at distal end 14. Proximal end surface 66 of valve element 46 may be shaped and configured to be substantially coplanar and level with proximal end 14 of connector element 12 when valve element 46 is in the closed state. This substantially coplanar and level orientation is preferred in order to avoid reservoirs or crevices between proximal end surface 66 of valve element 46 and proximal end 14 of connector element 12 in which pooling of fluid and/or debris and/or bacterial contamination may result. Furthermore, the substantially coplanar and level orientation allows for an easily swabable surface for disinfection.

Furthermore, in the closed state, shaft portion 56 of valve element 46 is located in axial bore 20 of connector element 12 and interior 38 of container insert member 28. Preferably, distal end 50 of valve element 46 is rounded and, as mentioned earlier, rests on or abuts the distal potion 31 of interior 38. Also, the distal portion of valve element 46 can move laterally with respect to connector element 12 and container insert member 28 while the proximal portion of valve element 46, as a result of the relative smaller size of bore 20 with respect to interior 38, can move laterally to a lesser degree, if at all, with respect to connector element 12 and container insert member 28.

As illustrated in FIG. 7, the open state of fluid flow control device 10 is achieved by compressing valve element 46 into container insert member 28 generally along axis 52. This compression may be achieved in a preferred embodiment by connecting a common male Luer fitting 70 to proximal portion 19 of connector element 12. By providing control device 10 with characteristics for being actuated by a common Luer fitting 70, there is no need for a needle or similarly sharp instrument to access and to withdraw fluid from fluid container 41. Of course, other means of compression are also contemplated, including using a blunt, elongated member to displace valve element 46.

In FIG. 7, valve element 46 is compressed into container insert member 28 along axis 52. As used herein, the word “compress” and its derivatives refers to any position wherein the two ends of an elongate element are brought closer together. This may occur due to a shortening of the element along its axial length or due to a bending of the element off-axis which may or may not be accompanied by a shortening in the length of the element.

As the male Luer fitting 70 is inserted into axial bore 20, it initially contacts proximal end surface 66 of valve element 46. Further insertion of fitting 70 places an axial load on valve element 46 which is distributed along the entire length of valve element 46. In a preferred embodiment, this compressive force operates initially to break the secondary seal between nose 64 and axial bore 20, and then to compress plug portion 54 primarily at the buckling section formed by notch 68.

As plug portion 54 collapses at notch 68, plug portion 54 compresses along the side of valve element 46 at which notch 68 is formed, causing proximal end surface 66 to dip in the area of notch 68. This effectively moves the area of contact between the Luer fitting 70 and proximal end surface 66 away from axis 52 and toward the side of plug portion 54 opposite notch 68. This movement displaces a portion of valve element 46 away from axis 18 of connector element 12 in a first direction.

As valve element 46 collapses along notch 68, the additional force applied by insertion of fitting 70 may also operate to compress shaft portion 56 axially. With the compression of valve element 46, the shaft portion 56 tends to cant, buckle, bow or otherwise bend (“cant” or “canting” hereafter for the sake of simplicity and without the intent to limit). This compression of shaft portion 56 tends to break the primary seal formed between surface 60 of plug portion 54 and valve seat 26 of connector element 12. This movement of shaft portion 56 also displaces a portion of valve element 46 away from axis 34 of container insert member 28 in a second direction. In an exemplary embodiment, the displacement of a portion of a valve element 46 in the first direction may be in the same direction as the displacement of a portion of valve element 46 in the second direction. In another exemplary embodiment, the displacement of a portion of valve element 46 in the first direction may be in a different direction from the displacement of a portion of valve element 46 in the second direction. Also, distal end 57 of shaft 56 may slide along the rounded distal portion of interior 38 of container insert member 28 to facilitate canting and compression of shaft portion 56. Having broken both the primary seal associated with the primary valve seat 26, and the secondary seal associated with nose 64, the channel is now opened to allow fluid to flow from the interior of container 41 to fitting 70. In an exemplary embodiment, the fluid flows from the interior of container 41 through windows 42 to interior 38. From interior 38, the fluid flows through opening 36 into axial bore 20, around valve element 46, and into fitting 70.

Turning now to FIG. 8, a container wrapper member 72 is shown in isolation. In an exemplary embodiment, container wrapper member 72 may also be included to secure flange 44 of container insert member 28 to lip 45 of container 41. Container wrapper member 72 may be coupled to flange 44 and lip 45 in a number of ways including bonding agents, ultrasonic welding, interference fitting, and tongue and groove fittings.

While various descriptions of the present invention are described above, it should be understood that the various features can be used singly or in combination thereof. Therefore, this invention is not to be limited to the specific preferred embodiments depicted herein. Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A needleless access fluid container adapted to facilitate flow between said fluid container and a needleless fluid extraction device having a male Luer fitting, said needleless access container comprising:
a fluid container having an opening;
a control device having a channel, a proximal end, a distal end, and a first axis extending between said proximal end and said distal end, said proximal end of said connector being capable of coupling to the fluid extraction device, and said distal end of said connector being coupled to said opening of said fluid container;
a connector element forming a proximal portion of said control device;
a container insert member forming a distal portion of said control device and having a plurality of windows extending from said distal end of said connector toward said proximal end of said connector and fluidly connecting said channel to said interior of said container; and
a valve element disposed in said channel of said control device and having a plug and a shaft, said plug and said shaft biasing said valve element into sealing engagement with said connector to seal said channel in a first position of said valve element, and said plug and said shaft being axially compressible to displace said valve element from said connector to open said channel in a second position of said valve element;
wherein insertion of the male Luer fitting of the fluid extraction device into said proximal end of said control device moves said valve element from said first position to said second position, opening said channel for fluid flow between said container and said fluid extraction device.

2. The needleless access fluid container of claim 1, wherein said connector further comprises a flange for abutting a lip surrounding said opening of said container.

3. The needleless access fluid container of claim 1, wherein said connector is configured for coupling to the fluid extraction device near said proximal end of said connector.

4. A fluid flow control device configured for connecting to a fluid container, said fluid flow control device comprising:
a container insert member having a proximal end, a distal end, and a first axis extending therebetween;
a connector element defining a valve seat coupled to said container insert member, and having a proximal end with a first swabable surface, a distal end, and a second axis extending therebetween; and
a resilient valve element disposed in said container insert element and said connector element, being movable from a first position to a second position, and defining a proximal end having a second swabable surface;
wherein:
in said first position said valve element forms a seal with said valve seat and is substantially aligned with at least one of said first axis of said container insert member and second axis of said connector element; and
in said second position said valve element is compressed and moved from said valve seat and is displaced from said at least one of said first axis of said container insert member and second axis of said connector element to create a fluid channel.

5. The control device of claim 4, wherein:
said container insert member has a hollow interior defining an opening extending from a proximal surface of said container insert member to said interior; and
at least one window extends from an outer surface of said container insert member to said interior.

6. The control device of claim 5, wherein said at least one window extends along said container insert member from said distal end of said container insert member toward said proximal end of said container insert member.

7. The control device of claim 5, wherein said container insert member further comprises a flange located near said proximal end of said container insert member for abutting a lip surrounding a fluid container opening.

8. The control device of claim 5, wherein:
said distal end of said container insert member is substantially rounded; and
a distal end of said hollow interior is substantially rounded.

9. The control device of claim 5, wherein said opening on said proximal surface of said container insert member is surrounded by a collar.

10. The control device of claim 4, wherein a portion of said container insert member supports said valve element only axially and not laterally to prevent axial movement of said valve element so that said valve element cants upon exertion of pressure on said proximal end thereof.

11. The control device of claim 4, wherein said connector element has an axial throughbore extending from said proximal end to said distal end and further comprises:
a collar located at said distal end of said connector element for coupling to said proximal end of said container insert member.

12. The control device of claim 11, wherein a portion of said axial bore forms said valve seat.

13. The control device of claim 11, wherein said connector element is configured for coupling with a fluid extraction device near said proximal end of said connector element.

14. The control device of claim 13, wherein said connector element is configured for coupling with a male Luer fitting.

15. The control device of claim 14, wherein coupling of a male Luer fitting to said proximal end of said connector element moves said valve element from said first position to said second position, forming a fluid channel between said fluid container and the male Luer fitting of the fluid extraction device.

16. The control device of claim 4, wherein said valve element further comprises:
a plug having a proximal end, a distal end, and a first portion of a third axis extending therebetween and disposed near said proximal end of said connector element; and
a shaft formed at said distal end of said plug having a proximal end, a distal end, and a second portion of said third axis extending therebetween and extending to said distal end of said container insert member.

17. The control device of claim 16, wherein said shaft is integral with said plug.

18. The control device of claim 16, wherein said distal end of said shaft is rounded.

19. The control device of claim 16, wherein:
a portion of said plug forms at least one buckling section with characteristics for being axially compressible to move said valve element from said first position to said second position; and
said shaft forms at least one buckling section with characteristics for being axially compressible to move said valve element from said first position to said second position.

20. The control device of claim 16, wherein said first portion of said valve element is displaced from said first axis in a first direction and said second portion of said valve element is displaced from said second axis in a second direction, wherein said first direction and said second direction are the same direction.

21. The control device of claim 16, where said first portion of said valve element is displaced from said first axis in a first direction and said second portion of said valve element is displaced from said second axis in a second direction wherein said first direction is a different direction than said second direction.

22. The control device of claim 4, wherein said first swabable surface of said connector element and said second swabable surface of said valve element are substantially co-planar.

24. The control device of claim 4, wherein a first portion of said valve element is displaced from said first axis of said container insert member in a first direction and a second portion of said valve element is displaced from said second axis of said connector element in a second direction.

25. The control device of claim 7, further comprising a container wrapper member configured and dimensioned to secure said flange of said container insert member to said container.

26. A method for using a needleless access fluid container, comprising:
providing a connector at an opening of said fluid container, the connector having a connecting element at a proximal portion of the connector and a container insert member at a distal portion of the connector, the connecting element and the container insert member defining a channel and an axis, the connector further including a resilient valve element in said channel of the connector, the resilient member having a rounded distal surface and at least one notch to facilitate canting, and being biased to close the channel of the connector;
engaging the proximal end of the connector with a male fitting of a fluid extraction device; and
inserting the male fitting into the proximal end of the connector to cause the valve element to cant, thereby opening the channel to allow fluid communication between the container and the fluid extraction device.

27. A fluid flow control device configured for connecting to a fluid container, said fluid flow control device comprising:
a container insert member having a proximal end, a distal end, and a first axis extending therebetween;
a connector element defining a valve seat coupled to said container insert member, and having a proximal end, a distal end, and a second axis extending therebetween; and
a resilient valve element disposed in said container insert element and said connector element, being movable from a first position to a second position;
wherein:
in said first position said valve element forms a seal with said valve seat and is substantially aligned with at least one of said first axis of said container insert member and said second axis of said connector element;
in said second position said valve element is compressed and moved from said valve seat and is displaced from said at least one of said first axis of said container insert member and said second axis of said connector element to open a fluid channel extending through said fluid flow control device and in communication with the interior of the fluid container; and
a portion of said container insert member supports said valve element only axially and not laterally to prevent axial movement of said valve element so that said valve element cants upon exerted pressure on said proximal end thereof.

28. The control device of claim 27, wherein a portion of said valve element forms at least one buckling section in the form of a notch with characteristics for being axially compressible.

29. The control device of claims 27, further comprising an “o”-ring positioned between said flange of said container insert member and the container.
